(19) United States
(12) Patent Application Publication
(10) Pub. No.: US 2024/0021289 A1
(43) Pub. Date: Jan. 18, 2024

(54) Combining Dietary Recommendations Across Comorbidities

(71) Applicant: Lisa Marie D'Agostino, Boca Raton, FL (US)

(72) Inventor: Lisa Marie D'Agostino, Boca Raton, FL (US)

(21) Appl. No.: 18/221,795

(22) Filed: Jul. 13, 2023

Related U.S. Application Data
(60) Provisional application No. 63/368,771, filed on Jul. 18, 2022.

(51) Int. Cl. G16H 20/60

(52) U.S. Cl. CPC G16H 20/60; G16H 70/20

(57) ABSTRACT

The system outlined herein utilizes a unique code that allows the inventor to combine dietary recommendations for multiple comorbities. The method allows the user to enter multiple medical ailments from a list and generates food items as either “allowed” (in green font) or “not allowed” (in red font) based on pre-set dietary restrictions for each of the ailments that were selected. This is the first electronic platform that allows the user to receive dietary recommendations for multiple comorbidities in one place. The proprietary system generates a different kind of database that gives the system the ability to provide dietary recommendations for multiple ailments at one time. Once the comorbidities are selected, the system generates a list of foods that the user is permitted to consume based on the most up-to-date dietary recommendations for each of their ailments.

### CROSS-REFERENCE

Provisional patent application #63/368,771

### BACKGROUND OF THE INVENTION

The present invention relates to the field of technology. Specifically, the present invention relates to an application that utilizes a system to combine the dietary recommendations for multiple ailments onto one electronic platform.

Healthcare professionals are faced with the task of helping people make healthy lifestyle choices to positively impact their medical status. This involves taking into consideration each patient's complex lists of comorbidities. Comorbidities is defined as the simultaneous presence of two or more diseases or medical conditions in an individual. I will use this term interchangeably with “ailments,” “diagnoses,” and “diseases” in the following sections.

On a daily basis, medical professionals tell patients what they should and should not eat to prevent further complications associated with medical ailments ranging from hypertension and diabetes to liver cirrhosis and exocrine pancreatic insufficiency (EPI); the list goes on. Once they finish going through an exhaustive list of foods that the patient must avoid, the patient's general response is a snicker of defeat and the question, “so what can I eat?”

Unfortunately, in the healthcare industry today, there is not enough time in the day to answer this complex question properly during the brief time they have to spend with patients during a regular office visit. Referring patients to a registered dietitian is always an option, but one that oftentimes comes at a high cost and is not covered by most major insurance companies.

In today's fast-paced world, more and more people turn to web or mobile phone applications for help with their activities of daily living, including shopping and dietary management. The services available presently hone in on only one medical ailment at a time or focus on weight loss measures. There are currently no platforms that allow consumers to combine their comorbidities in a way that follows best practice guidelines for dietary management of their multiple ailments. What is needed are techniques that overcome these disadvantages.

### BRIEF SUMMARY OF THE INVENTION

The following sections explore a new dietary system, also referred to throughout this document as an application. We will explore the unique techniques it utilizes in aiding consumers and patients with a lifelong task that significantly impacts their health. This unique web-application makes it easy for an individual to tailor their dietary needs in a way that meets best practice guidelines for management of their multiple ailments. What makes the code for this electronic platform so unique is it's ability to combine dietary recommendations across multiple diagnoses. It is not a weight loss app, nor does it pose the restrictions of dietary modifications for a single ailment. It is all encompassing, and finally helps answer every patient's question, “what can I eat?”

This unique system individualizes dietary recommendations by using a unique code, which combines the dietary parameters for two or more comorbidities. Services today focus on weight loss or provide dietary recommendations for a single ailment. None have taken all of a patient's diagnoses and dietary restrictions into consideration on one easy-to-use platform.

### BRIEF DESCRIPTION OF THE DRAWINGS

FIG. 1 illustrates the welcome and demographics page, according to one embodiment of the present invention.

FIG. 2 illustrates the comorbidities page, according to one embodiment of the present invention.

FIG. 3 illustrates the search bar and results page, according to one embodiment of the present invention.

FIG. 4 illustrates the result details page, according to one embodiment of the present invention.

FIG. 5 illustrates an example of a user's shopping list, according to one embodiment of the present invention.

FIG. 6 illustrates the application's unique code.

Various embodiments and aspects of the inventions will be described with reference to details discussed below, and the accompanying drawings will illustrate the various embodiments. The following description and drawings are illustrative of the invention and are not to be construed as limiting the invention.

Reference in the specification to “one embodiment” or “an embodiment” or “another embodiment” means that a particular feature, structure, or characteristic described in conjunction with the embodiment can be included in at least one embodiment of the invention. The appearances of the phrase “in one embodiment” in various places in the specification do not necessarily all refer to the same embodiment.

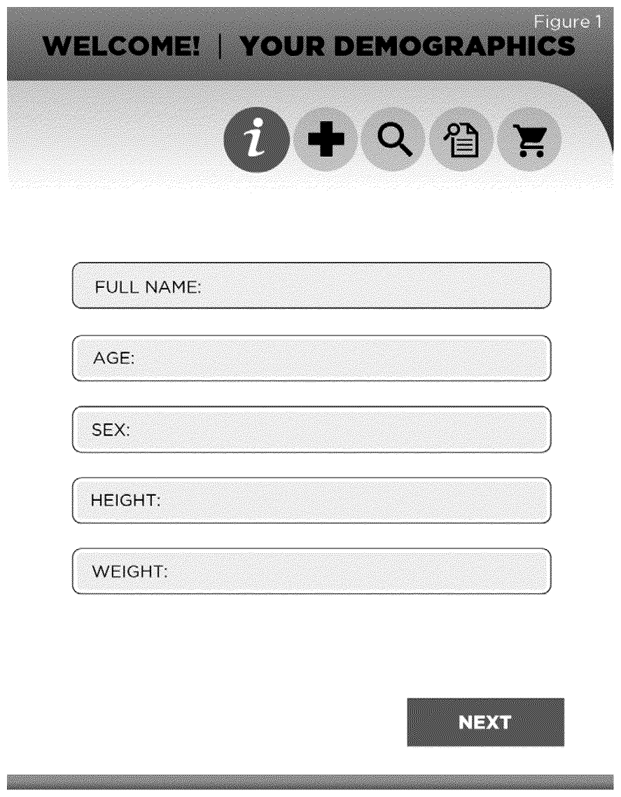

FIG. 1 illustrates an embodiment of the welcome page for the application, where the user enters their basic demographic information. Across the top of the figure you can see the menu. The “i” indicates user information and can be accessed and edited at any time.

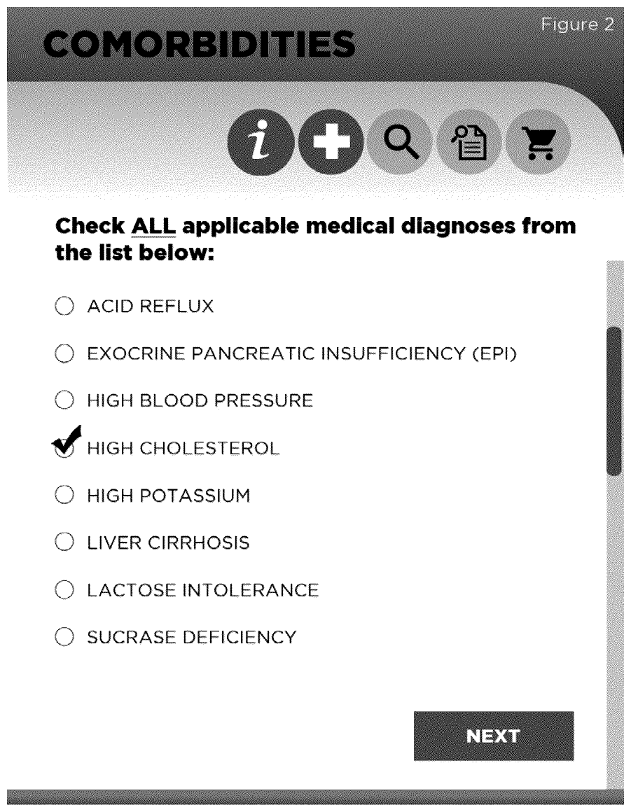

FIG. 2 illustrates an embodiment of the second page of the application that the user will see after entering their demographics on the welcome page and clicking “next”. In this example, the user has selected “high cholesterol.” For simplicity explaining in-app functions, we are only selecting one comorbidity. The user is, of course, not limited to the number of comorbidities that they may select in the real application. Across the top of the application, you can see that the medical “+” sign is now red, indicating the patient's comorbidities or ailments. This section can be accessed and edited at any time.

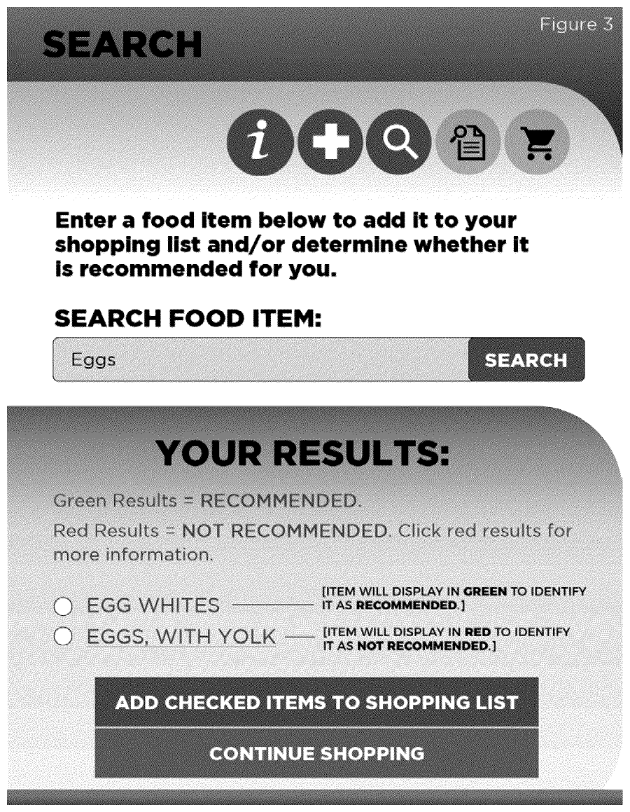

FIG. 3 illustrates an embodiment of the search page that the user sees after selecting their comorbidities on the prior screen and clicking “next”. In this example, the user types “eggs” into the search bar. Below that are their search results. Since eggs (with the yolk) would not be recommended for a patient with high cholesterol, that item is listed in red. The item listed in green is a helpful alternative that the user may choose to add to their shopping list instead. You can see a magnifying glass at the top of the screen. It can be selected at any time to be returned to the search page.

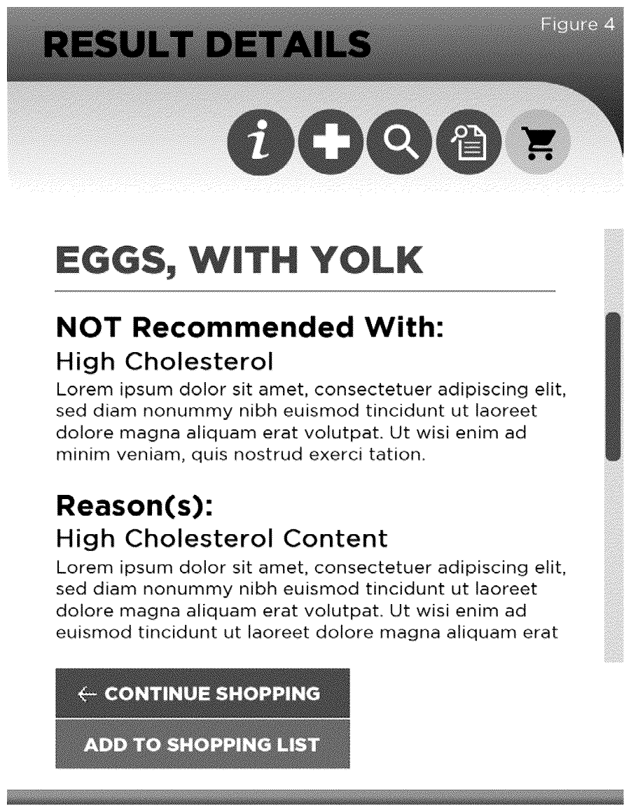

FIG. 4 illustrates an embodiment of the result details page, which the user accesses by clicking on a food item that was listed in red on the prior screen. Here, the user can see why that food item was not recommended. The explanation pertaining to “High Cholesterol” has been replaced with placeholder text in FIG. 4. All of the patient's selected comorbidities, for which this food is not recommended, are listed here with an explanation. From this screen, they can choose to add the item (despite not being recommended by the system) to their shopping cart, or click “continue shopping” to return to the search page.

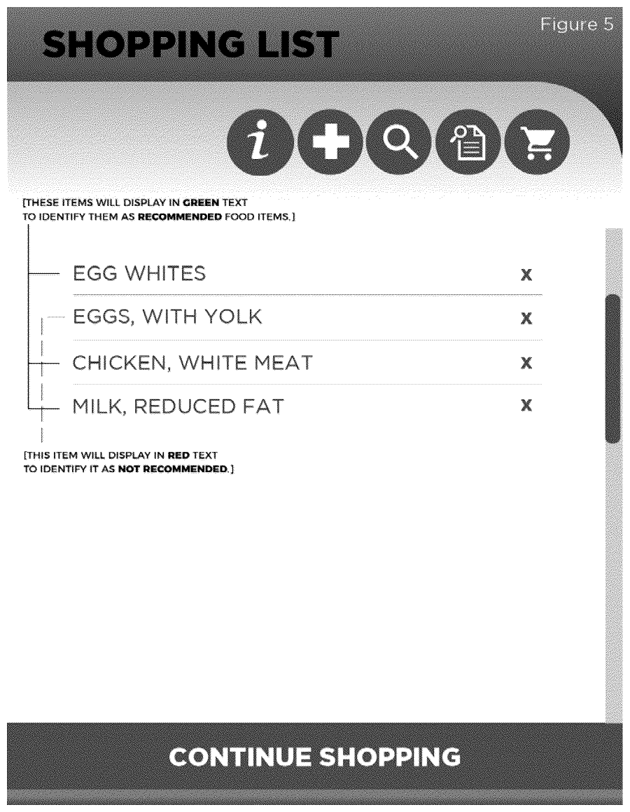

FIG. 5 illustrates an embodiment of a user's shopping list within the application. They have chosen to select food items that are recommended for their indicated ailments, along with ones that are not. The shopping cart icon at the top of the screen can be selected at any time to view the shopping list. The list can be edited by selecting the red “x” beside items that the user would like to remove.

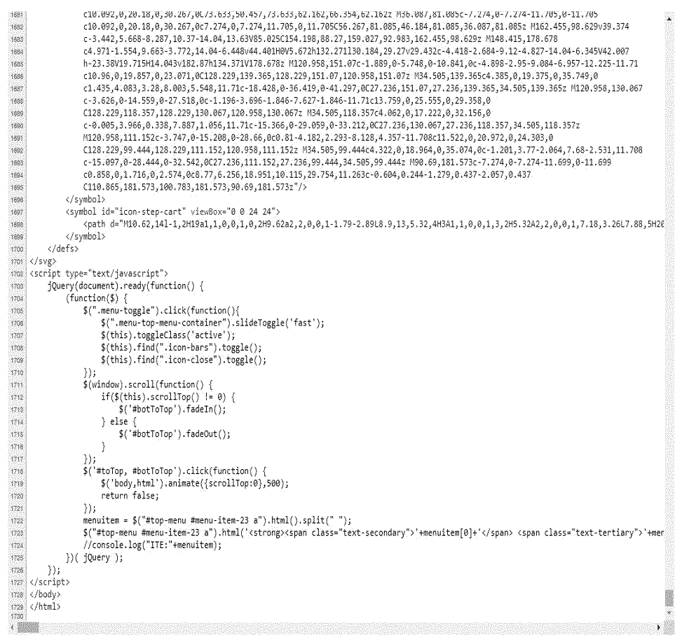

FIG. 6 illustrates the application's unique code. This enables the functions outlined herein and also expresses the embodiments of the platform outlined in FIGS. 1-5 referenced in this section. This unique code was created in the development of a web-based platform that combines dietary parameters for multiple comorbidities. It allows the inventor to combine said parameters across multiple illnesses in order to generate a single “allowed” or “not allowed” (indicated by green or red font, respectively) for each food item in a food database. The dietary parameters are pre-determined and entered by the inventor, and this code allows said parameters to be edited at any time in order to reflect the most up-to-medical recommendations.

### DETAILED DESCRIPTION OF THE INVENTION

Upon accessing the system, the user is taken to a home screen, where they are asked to provide basic demographic information. On the following screen, they simply check off all applicable medical diagnoses or comorbidities from the list provided.

The user is then taken to the search bar, where they can look up any food item. A simple example would be that the user has selected “high cholesterol” as one of their medical diagnoses, and then typed “eggs” into the search bar. Once they click search, “Eggs, with yolk” is listed below the search bar in red, indicating that the food item is not recommended for high cholesterol. Clicking on the red food item takes them to the next screen, which gives them an explanation as to why the food item is not recommended for the indicated medical diagnosis. Keep in mind, if the same food item were to not satisfy the user's dietary needs because of multiple ailments they indicated on the comorbidities page, then all of those ailments would be listed with their own explanation under that food item.

At this point, the user is also given the option to either add the item to their shopping list anyway, or “Continue Shopping.” If they click “Continue Shopping,” the item is not added to their shopping cart, and the user is brought back to the prior search where they can see “Egg whites” is listed in green. Green indicates a healthy alternative. They will hopefully choose to place the healthier alternative into their cart and continue building their shopping list in this fashion.

As it pertains to the proprietary code utilized by this platform, best-practice guidelines and dietary recommendations for all of the comorbidities indicated by the user's checked items (reference paragraph 15) are included. The software was designed to utilize pre-set parameters for each comorbidity in order to generate dietary recommendations. For example, if an individual selects diseases that require control of both their sodium as well as fat intake, then the system utilizes pre-set parameters, in order to generate a list of low sodium and low fat foods. The pre-set parameters referenced here are entered and modified by the inventor based on the most up-to-date recommendations in the medical field.

1. A method or system for making dietary recommendations comprising:
dietary parameters for various comorbities; and
said recommendations are generated by combining said parameters for two or more comorbidies or diseases.

2. The method or system of claim 1, further comprising a database, which is used to generate dietary recommendations based on pre-set parameters.

3. The database of claim 2, wherein health/dietary parameters are entered for multiple comorbidities in order to generate dietary recommendations based on an individuals' pre-selected comorbidities.
